(19) United States
(12) Patent Application Publication
(10) Pub. No.: US 2016/0331434 A1
(43) Pub. Date: Nov. 17, 2016

(54) SYSTEM AND METHODS FOR ABLATING TISSUE

(71) Applicant: VytronUS, Inc., Sunnyvale, CA (US)

(72) Inventors: Patrick Phillips, Los Altos, CA (US); John P. Madden, Emerald Hills, CA (US); Nicholas Patronik, San Jose, CA (US); Michael J. Horzewski, San Jose, CA (US)

(21) Appl. No.: 15/151,894

(22) Filed: May 11, 2016

Related U.S. Application Data
(60) Provisional application No. 62/159,531, filed on May 11, 2015.

(51) Int. Cl. A61B 18/04; A61B 34/20

(52) U.S. Cl. CPC A61B 18/04; A61B 34/20; A61B 2018/00577

(57) ABSTRACT

Tissue ablation systems and methods for creating ablation zones in tissue comprises locating a target tissue and positioning an interventional catheter adjacent the target tissue. The interventional catheter has an energy source. Collateral tissue adjacent the target tissue is located. The target tissue is ablated with energy from the energy source so as to form a continuous lesion or region of ablation in the target tissue. The ablating is conducted so as to minimize ablating or otherwise damaging the collateral tissue and to increase the likelihood of procedure success.

### CROSS-REFERENCE TO RELATED APPLICATIONS

The present application is a non-provisional of, and claims the benefit of U.S. Provisional Patent Application No. 62/159,531 (Attorney Docket No. 31760-725.101) filed May 11, 2015; the entire contents of which are incorporated herein by reference.

### BACKGROUND OF THE INVENTION

1. Field of the Invention

The present application generally relates to systems and methods for creating ablation zones in human tissue. More specifically, the present application relates to the treatment of electrophysiologic disorders of the heart by using ultrasound energy, and even more specifically, the present application relates to ablation systems and methods used to treat atrial fibrillation, ventricular tachycardia, periventricular contractions, etc. that detect and compensate for collateral tissue such as the phrenic nerve, esophagus, and other tissue.

The condition of atrial fibrillation (AF) is characterized by the abnormal (usually very rapid) beating of the left atrium of the heart which is out of synch with the normal synchronous movement (‘normal sinus rhythm’) of the heart muscle. In normal sinus rhythm, the electrical impulses originate in the sino-atrial node (‘SA node’) which resides in the right atrium. The abnormal beating of the atrial heart muscle is known as ‘fibrillation’ and is caused by electrical impulses originating instead at points other than the SA node, for example, in the pulmonary veins (PV).

There are pharmacological treatments for this condition with varying degree of success. In addition, there are surgical interventions aimed at removing the aberrant electrical pathways from PV to the left atrium (‘LA’) such as the ‘Cox-Maze III Procedure’. This procedure has been shown to be 99% effective but requires special surgical skills and is time consuming. Thus, there has been considerable effort to copy the Cox-Maze procedure using a less invasive percutaneous catheter-based approach. Less invasive treatments have been developed which involve use of some form of energy to ablate (or kill) the tissue surrounding the aberrant focal point where the abnormal signals originate in PV. The most common methodology is the use of radio-frequency (‘RF’) electrical energy to heat the muscle tissue and thereby ablate it. The aberrant electrical impulses are then prevented from traveling from PV to the atrium (achieving the ‘conduction block’) and thus avoiding the fibrillation of the atrial muscle. Other energy sources, such as microwave, laser, and ultrasound have been utilized to achieve the conduction block. In addition, techniques such as cryoablation, administration of ethanol, and the like have also been used.

More recent approaches for the treatment of AF involve the use of ultrasound energy. The target tissue of the region surrounding the pulmonary vein is heated with ultrasound energy emitted by one or more ultrasound transducers.

When delivering energy to tissue, in particular when ablating tissue with ultrasound to treat atrial-fibrillation, a transmural lesion (burning all the way through the tissue) must be made to form a proper conduction block. Achieving a transmural lesion though has many challenges. When ablating in the ventricle, it may or may not be desirable to create a transmural lesion, but similar concerns remain with respect to collateral tissue. Health complications may arise when esophageal or other collateral tissue such as the phrenic nerve is ablated. Thus there is a need in the medical device field to provide an ablation system and method of use that enables any lesion to be created, appropriately delivers energy to achieve conduction block, and that detects and enables compensation for collateral tissue as part of the procedure. It would also be desirable to provide an ablation system that is easy to use, enables capabilities not present in current therapies, and achieves greater efficacy and safety.

2. Description of the Background Art

Patents related to the treatment of atrial fibrillation include, but are not limited to the following: U.S. Pat. Nos. 8,224,422; 6,997,925; 6,996,908; 6,966,908; 6,964,660; 6,955,173; 6,954,977; 6,953,460; 6,949,097; 6,929,639; 6,872,205; 6,814,733; 6,780,183; 6,666,858; 6,652,515; 6,635,054; 6,605,084; 6,547,788; 6,514,249; 6,502,576; 6,416,511; 6,383,151; 6,305,378; 6,254,599; 6,245,064; 6,164,283; 6,161,543; 6,117,101; 6,064,902; 6,052,576; 6,024,740; 6,012,457; 5,405,346; 5,314,466; 5,295,484; 5,246,438; and 4,641,649.

Patent Publications related to the treatment of atrial fibrillation include, but are not limited to International PCT Publication No. WO 99/02096; and U.S. Patent Publication No. 2005/0267453. Scientific publications related to the treatment of atrial fibrillation include, but are not limited to: Haissaguerre, M. et al., Spontaneous Initiation of Atrial Fibrillation by Ectopic Beats Originating in the Pulmonary Veins, New England J Med., Vol. 339:659-666; J. L. Cox et al., The Development of the Maze Procedure for the Treatment of Atrial Fibrillation, Seminars in Thoracic & Cardiovascular Surgery, 2000; 12: 2-14; J. L. Cox et al., Electrophysiologic Basis, Surgical Development, and Clinical Results of the Maze Procedure for Atrial Flutter and Atrial Fibrillation, Advances in Cardiac Surgery, 1995; 6: 1-67; J. L. Cox et al., Modification of the Maze Procedure for Atrial Flutter and Atrial Fibrillation. II, Surgical Technique of the Maze III Procedure, Journal of Thoracic & Cardiovascular Surgery, 1995; 110:485-95; J. L. Cox, N. Ad, T. Palazzo, et al. Current Status of the Maze Procedure for the Treatment of Atrial Fibrillation, Seminars in Thoracic & Cardiovascular Surgery, 2000; 12: 15-19; M. Levinson, Endocardial Microwave Ablation: A New Surgical Approach for Atrial Fibrillation; The Heart Surgery Forum, 2006; Maessen et al., Beating Heart Surgical Treatment of Atrial Fibrillation with Microwave Ablation, Ann Thorac Surg 74: 1160-8, 2002; A. M. Gillinov, E. H. Blackstone and P. M. McCarthy, Atrial Fibrillation: Current Surgical Options and their Assessment, Annals of Thoracic Surgery 2002; 74:2210-7; Sueda T., Nagata H., Orihashi K., et al., Efficacy of a Simple Left Atrial Procedure for Chronic Atrial Fibrillation in Mitral Valve Operations, Ann Thorac Surg 1997; 63:1070-1075; Sueda T., Nagata H., Shikata H., et al.; Simple Left Atrial Procedure for Chronic Atrial Fibrillation Associated with Mitral Valve Disease, Ann Thorac Surg 1996; 62:1796-1800; Nathan H., Eliakim M., The Junction Between the Left Atrium and the Pulmonary Veins, An Anatomic Study of Human Hearts, Circulation 1966; 34:412-422; Cox J. L., Schuessler R. B., Boineau J. P., The Development of the Maze Procedure for the Treatment of Atrial Fibrillation, Semin Thorac Cardiovasc Surg 2000; 12:2-14; and Gentry et al., Integrated Catheter for 3-D Intracardiac Echocardiography and Ultrasound Ablation, IEEE Transactions on Ultrasonics, Ferroelectrics, and Frequency Control, Vol. 51, No. 7, pp 799-807.

### SUMMARY OF THE INVENTION

The present application generally relates to systems and methods for creating ablation zones in human tissue. More specifically, the present application relates to the treatment of electrophysiologic disorders of the heart by using ultrasound energy with ablation systems and methods that detect and compensate for collateral tissue such as the esophagus, phrenic nerve, bronchi, and other tissue.

In a first aspect of the present invention is a tissue ablation method for treating atrial fibrillation in a patient comprises positioning an interventional catheter within a cardiac chamber and locating a target tissue, such as around one or more pulmonary veins, a roof line, and isthmus line, a zone of ablation (for treating rotors), etc. The interventional catheter has an energy source. Collateral tissue adjacent the cardiac chamber is identified. A lesion path is identified in the target tissue. The target tissue is transmurally ablated with energy from the energy source without contact between the energy source and the target tissue and preferably while the energy source is moving. This forms a continuous transmural lesion or an ablation zone within the target tissue and the lesion blocks aberrant electrical pathways in the tissue so as to reduce or eliminate the atrial fibrillation. The lesion path and/or the energy dose profile delivered to the target tissue is selected and/or adjusted so as to minimize ablating or otherwise damaging the collateral tissue and/or to ensure transmurality of the lesion. A portion of the cardiac chamber, the lesion path, tissue information, system information, and/or the collateral tissue may be shown on a display in 2D or 3D. The display may be part of the energy delivery system or a stand-alone display.

The interventional catheter may further comprise a sensor adjacent the energy source. Locating may comprise delivering energy from the energy source toward the tissue adjacent the target tissue, and sensing energy reflected from the tissue adjacent the target tissue with the sensor. The sensor may comprise at least a portion of the energy source. The catheter may further incorporate one or more coils to assist in determining position of a portion of the catheter within the patient, when used with a mapping system (e.g. electromagnetic mapping system). The mapping system may be part of the energy delivery system console, console-integrated, or a stand-alone system.

Positioning may comprise intravascularly advancing the interventional catheter into a left or right atrium of the patient's heart. Identifying may comprise characterizing properties of the tissue and comparing the properties with known tissue properties. Identifying may be a part of a diagnostic sweep of the target tissue, tissue adjacent the target tissue, and collateral tissue with or without contacting the target tissue. The sweep may be a systematic scan to acquire information about the tissue adjacent the target tissue. Identifying may be performed before or while the ablating step is performed.

The modifying may comprise modifying the transmural lesion so as to avoid the collateral tissue. Modifying may comprise changing an originally planned transmural lesion path to a new transmural lesion path and/or modifying may comprise changing the energy dose profile delivered to the target tissue so as to minimize damaging the collateral tissue.

The collateral tissue may comprise an esophagus. Identifying the esophagus may comprise positioning a detection device into the esophagus. Identifying may also comprise sensing the presence of the detection device through one or more layers of tissue. The detection device may comprise a tubular member which may have a lumen extending at least partially through or within the detection device. The detection device may contain a balloon. The lumen and/or balloon may be filled with a fluid such as saline, water, gas (e.g. carbon dioxide, air). Liquids such as saline or water are preferably filled with microbubbles to enhance echogenicity. The method may further comprise sensing the filled lumen or balloon with an ultrasound signal delivered by the energy source. The detection device may also comprise a transponder such as a reflective material, a chemical substance, RFID tag, a capacitive plate, an inductive component, an ultrasound transducer, and an infrared light. The detection device may comprise one or more coils within the detection device that is identified by an electromagnetic component, such as a window field generator, mapping system, and/or the ablation system. The detection device may further protect the esophagus by cooling the esophagus. Identifying the esophagus may comprise sensing the detection device with the interventional catheter and/or with the electromagnetic system and/or the ablation system. A portion of the cardiac chamber, the lesion path, and/or the esophagus may be shown on a display in 2D or 3D. The display may be part of the energy delivery system or a stand-alone display. The detection device may comprise a temperature monitoring component or components such as one or more thermistors or thermocouples. The temperature monitoring component(s) may be used, for example, to monitor the temperature of the esophagus before, during, and/or after energy delivery.

The collateral tissue may also comprise a phrenic nerve. Identifying the nerve may comprise applying pressure or an electrical signal to the phrenic nerve and monitoring the patient for a reflex response. The reflex response may comprise a hiccup. Monitoring may comprise audibly monitoring the patient. Applying pressure may comprise directing an ultrasound pressure wave to the phrenic nerve, pushing on the nerve with an instrument or electrically stimulating the nerve.

In another aspect of the present invention, an energy delivery system for treating atrial fibrillation in a patient comprises an interventional catheter having an energy source and a sensor. The energy source is adapted to deliver a beam of energy to tissue thereby ablating tissue around one or more pulmonary veins to form a continuous lesion circumscribing one or more pulmonary veins. The continuous lesion blocks aberrant electrical pathways in the tissue so as to reduce or eliminate the atrial fibrillation. The energy delivery system includes a console, display, and elements for the operator to control/command the various aspects of the energy delivery system, including but not limited to motion of portions of the interventional catheter, energy delivery, 2D and/or 3D display(s), etc. The energy delivery system also includes a detection device positionable and/or movable in the esophagus. The detection device may have one or more transponders or electromagnetic coil components detectable by the sensor through one or more layers of tissue.

The detection device may comprise a tubular member which may have a lumen extending at least partially through or within the detection device. The detection device may contain a balloon. The lumen and/or balloon may be filled with a fluid such as saline, water, gas (e.g. carbon dioxide, air). Liquids such as saline or water are preferably filled with microbubbles to enhance echogenicity. The energy delivery system may enable sensing the filled lumen or balloon with an ultrasound signal delivered by the energy source. The transponder may comprise one of a reflective material, a chemical substance, RFID tag, a capacitive plate, an inductive or coil component (electromagnetic), an ultrasound transducer, and an infrared light. The detection device may comprise one or more coils within the detection device that is identified by an electromagnetic component, such as a window field generator, or mapping system and/or the ablation system. The energy source may comprise an ultrasound transducer, and the sensor may comprise at least a portion of the ultrasound transducer.

The energy delivery system may automatically provide a recommended dose profile and also enables the operator changing the energy dose delivered to the target tissue which may include increasing or decreasing the acoustic power and/or the speed at which the ultrasound beam moves across the tissue. Where the ultrasound beam is directed such that collateral tissue may be damaged, the energy dose may be decreased to provide a level of safety. Where the ultrasound beam is directed such that collateral tissue may not be or is unlikely to be damaged, the energy dose may be increased to provide a greater level of achieving transmurality. A constant acoustic power and speed may also be specified as the dose profile.

In another aspect of the present invention, similar techniques to those described above may be used to create a zone of ablation in the right atrium as well as the ventricles (e.g. to treat ventricular tachycardia or other arrhythmias). The zone of ablation also need not be transmural. For example, in treating ventricular tachycardia, it may be sufficient to only ablate an electrical channel and/or a region of scar tissue to achieve the desired therapeutic effect.

In another aspect, a method for ablating tissue comprises providing a catheter having an ultrasound transducer adjacent a distal end thereof and a sensor adjacent the ultrasound transducer, positioning the ultrasound transducer and the sensor adjacent target tissue, and sensing collateral tissue adjacent the target tissue. The method also comprises determining a lesion path based on the target tissue and the collateral tissue and ablating the target tissue along the lesion path with a beam of ultrasound from the ultrasound transducer thereby forming a continuous lesion in the target tissue.

The ultrasound transducer and the sensor may be the same ultrasound transducer, and the method may comprise maintaining a gap between the ultrasound transducer and the target tissue so that the ultrasound transducer is not in contact with the target tissue during energy delivery. Sensing the collateral tissue may comprise identifying collateral tissue with the ultrasound transducer or the sensor, or sensing the collateral tissue may comprise placing an esophageal probe in the esophagus.

In any embodiment, the esophageal probe may comprise an electromagnetic coil or a plurality of electromagnetic coils, and the esophageal probe may be identified by the ultrasound transducer or sensor. The esophageal probe may be moved within the esophagus to obtain position information. The esophageal probe electromagnetic coil may act as a sensor that may be identified by a system.

In another aspect, a method for ablating tissue comprises providing an ablation system, the ablation system having mapping and ablation capability, and providing a catheter having an ultrasound transducer adjacent a distal end thereof and a sensor adjacent the ultrasound transducer. The method also comprises positioning the ultrasound transducer and the sensor adjacent target tissue, mapping the target tissue, determining a position of collateral tissue, determining a lesion path based on the target tissue and the collateral tissue, and ablating the target tissue along the lesion path with a beam of ultrasound from the ultrasound transducer thereby forming a continuous lesion in the target tissue.

The ultrasound transducer and the sensor may be the same ultrasound transducer. The method may comprise maintaining a gap between the ultrasound transducer and the target tissue so that the ultrasound transducer is not in contact with the target tissue during energy delivery.

The ablation system may comprise a graphical display and the method may comprise displaying information on the graphical display. The graphical display may show the target tissue, the collateral tissue, or the thickness of the target tissue.

The method may comprise determining a dose profile delivered to ablate the target tissue based on the target tissue thickness, the position of the collateral tissue, or the depth of the target tissue. Determining the position of the collateral tissue may comprise identifying the collateral tissue with the ultrasound sensor or the sensor. The sensing of the collateral tissue may comprise placing an esophageal probe in the esophagus, and the esophageal probe may be identified with the ultrasound transducer or the sensor.

The esophageal probe may contain an electromagnetic coil or a plurality of electromagnetic coils. The method may further comprise identifying the esophageal probe with the ablation system. The ablation system may comprise an electromagnetic positioning system.

In yet another aspect, a system for ablating tissue comprises an ablation system comprising a sensor adapted to detect collateral tissue adjacent the target tissue, and a catheter having an ultrasound transducer adjacent a distal end thereof. The sensor provides feedback to the ablation system to provide positional information related to the collateral tissue. The ultrasound transducer is adapted to deliver a beam of ultrasound energy to target tissue thereby ablating the target tissue and forming a continuous lesion in the target tissue.

The ultrasound transducer and the sensor may be the same ultrasound transducer. The beam of ultrasound energy may be a collimated beam of ultrasound energy. The ultrasound transducer may be disposed away from the target tissue such that the ultrasound transducer is not in contact with the target tissue during energy delivery.

The ablation system may comprise a graphical display. The graphical display may show the target tissue, the collateral tissue, or the thickness of the target tissue. A dose profile may be delivered to ablate the target tissue, and may be determined by the tissue thickness, the position of the collateral tissue, or a depth of the target tissue.

The ultrasound transducer or the sensor may be configured to identify the collateral tissue. The system may further comprise an esophageal probe that is configured to be disposed in the esophagus, and configured to facilitate sensing of the collateral tissue. The esophageal probe may contain a single electromagnetic coil or a plurality of electromagnetic coils. The esophageal probe may be identified by the ultrasound transducer. The electromagnetic coil or coils in the esophageal probe may be identified by the ablation system. The ablation system may comprise an electromagnetic system. The electromagnetic coil or coils in the esophageal probe may be identified by the electromagnetic system.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary embodiment of the energy delivery system.

FIG. 2 illustrates an exemplary embodiment of the catheter.

FIG. 3 illustrates an exemplary embodiment of the detection device.

### DETAILED DESCRIPTION OF THE INVENTION

Specific embodiments of the disclosed device, delivery system, and method will now be described with reference to the drawings. Nothing in this detailed description is intended to imply that any particular component, feature, or step is essential to the invention.

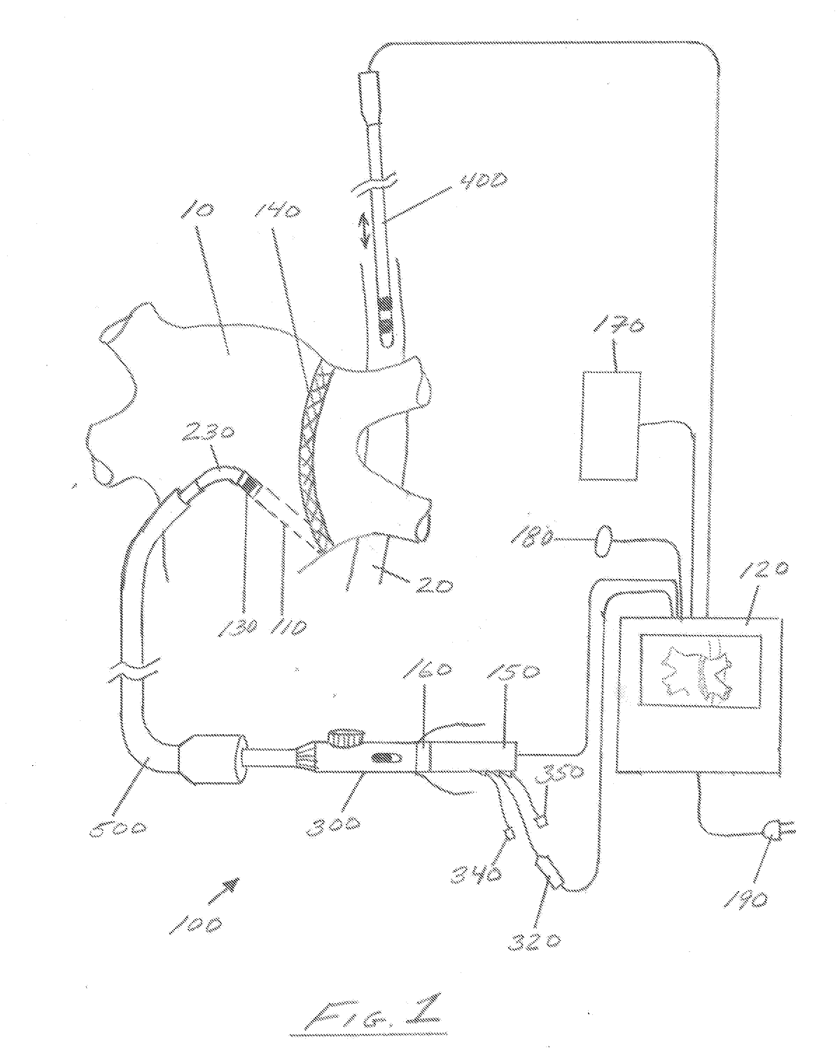

As shown in FIG. 1, the energy delivery system 100 of the preferred embodiments includes an energy source 210, which functions to provide a source of ablation energy that emits an energy beam 110. The energy delivery system 100 of the preferred embodiments also includes a sensor 220, or the energy source 210 may also serve as both the energy source and sensor as a single unit to detect the gap (distance of the target tissue surface from the energy source 210), the thickness of the tissue targeted for ablation, the characteristics of the ablated tissue, position and characteristics of collateral tissue, and any other suitable parameter or characteristic of the tissue and/or the environment around the energy delivery system 100. The energy source 210 is preferably moved and positioned within a patient, preferably within the left atrium 10 of the heart of the patient, such that the energy source 210 is positioned at an appropriate angle with respect to the target tissue. The angle is preferably any suitable angle such that the emitted energy beam 110 propagates into the target tissue, and preferably generates a transmural lesion, with resulting conduction block. Angles between 15 and 165 degrees are preferable because in this range the majority of the energy beam will preferably propagate into the tissue and the lesion depth needed to achieve transmurality is preferably minimally increased, though not required, from orthogonal. The energy delivery system 100 of the preferred embodiments also includes a console 120 operatively coupled to the energy source 210 and the sensor 220. The energy delivery system 100 is preferably designed for delivering energy to tissue, more specifically, for delivering ablation energy to tissue, such as heart tissue, to create a conduction block—isolation and/or block of conduction pathways of abnormal electrical activity, which typically originate from the pulmonary veins in the left atrium 10—for treatment of atrial fibrillation in a patient. The system 100, however, may be alternatively used with any suitable tissue in any suitable environment and for any suitable reason, such as for renal or other regions of denervation and ablation of cardiac tissue within the right atrium and ventricles.

As shown in FIG. 1, the distal tip 280 and the energy source 210 within it are preferably moved along a lesion path 140 such that the energy source 210 provides a partial or complete zone of ablation along the lesion path 140. The zone of ablation along the lesion path 140 preferably has any suitable geometry to provide therapy, such as providing a conduction block for treatment of atrial fibrillation in a patient. The zone of ablation along the lesion path 140 may alternatively provide any other suitable therapy for a patient. A linear zone of ablation is preferably created by moving the distal tip 280, and the energy source 210 within it, along an X, Y, and/or Z-axis.

As shown in FIG. 1, the energy delivery system 100 of the preferred embodiments may also include a catheter 200. The catheter 200 is shown positioned within a typical sheath 500 with the catheter 200 extending into a left atrium 10. The catheter 200 is preferably made of a flexible multi-lumen tube, but may alternatively be a cannula, tube or any other suitable elongate structure having one or more lumens. The catheter 200 of the preferred embodiments functions to accommodate pull wires, coils, deflecting and telescoping shafts, fluids, gases, energy delivery structures, electrical wires, therapy catheters, navigation catheters, pacing catheters, connections and/or any other suitable device or element. As shown in FIG. 1, the catheter 200 preferably includes a distal tip 280 positioned at a distal portion of the catheter 200. The catheter 200 further functions to move and position the energy source 210 and/or the distal tip 280 within a patient, such that the emitted energy beam 110 propagates into the target tissue at an appropriate angle as the energy source 210 and/or the distal tip 280 is moved along an lesion path 140 such that the energy source 210 provides a partial or complete zone of ablation along the lesion path 140.

The energy source 210 is preferably an ultrasound transducer that emits an ultrasound beam, but may alternatively be any suitable energy source that functions to provide a source of ablation energy. Suitable sources of ablation energy include but are not limited to, radio frequency (RF) energy, microwaves, photonic energy, and thermal energy. The therapy could alternatively be achieved using cooled sources (e.g., cryogenic fluid). The energy delivery system 100 preferably includes a single energy source 210, but may alternatively include any suitable number of energy sources 210. The ultrasound transducer is preferably made of a piezoelectric material such as PZT (lead zirconate titanate) or PVDF (polyvinylidine difluoride), or any other suitable ultrasound emitting material. For simplicity, the front face of the ultrasound transducer is preferably flat, but may alternatively have more complex geometry such as either concave or convex to achieve an effect of a lens or to assist in apodization—selectively decreasing the vibration of a portion or portions of the surface of the ultrasound transducer—and management of the propagation of the energy beam 110. The ultrasound transducer preferably has a circular geometry, but may alternatively be elliptical, polygonal, or any other suitable shape. The ultrasound transducer may further include coating layers which are preferably thin layer(s) of a suitable material. Some suitable ultrasound transducer coating materials may include graphite, metal-filled graphite, gold, stainless steel, magnesium, nickel-cadmium, silver, and a metal alloy. For example, the front face of the energy source 210 is preferably coupled to one or more matching layers. The matching layer(s) preferably functions to increase the efficiency of coupling of the energy beam 110 into the surrounding fluid. The matching layer is preferably made from a plastic such as parylene, preferably placed on the ultrasound transducer face by a vapor deposition technique, but may alternatively be any suitable material, such as graphite, metal-filled graphite, metals, or ceramic, added to the ultrasound transducer in any suitable manner.

The ultrasound transducer operates at a frequency preferably in the range of 5 to 25 MHz, more preferably in the range of 8 to 20 MHz, and even more preferably in the range of 8 to 15 MHz. The acoustic energy of the energy beam 110 is determined by the excitation voltage applied to the energy source 210, the duty cycle, and the total time the voltage is applied. The voltage is preferably in the range of 5 to 300 volts peak-to-peak. In addition, a variable duty cycle is preferably used to control the average power delivered to the energy source 210. The duty cycle preferably ranges from 0% to 100%, with a repetition frequency that is preferably faster than the time constant of thermal conduction in the tissue. One such appropriate repetition frequency is approximately 40 kHz. The average power level is preferably 0.5 to 25 watts and more preferably 2 to 15 watts, with power densities ranging from 50 watts/cm2 to 5000 watts/cm2.

When energized with an electrical signal or pulse train from the console 120, the energy source 210 emits an energy beam 110 (such as a sound pressure wave). The properties of the energy beam 110 are determined by the characteristics of the energy source 210 which determine the frequency, bandwidth, and amplitude of the energy beam 110 (such as a sound wave) propagated into the tissue. The energy source 210 emits an energy beam 110 such that it interacts with the target tissue along the lesion path 140 and forms a lesion. As the energy beam 110 travels through the target tissue, its energy is absorbed and scattered by the target tissue with most being converted to thermal energy. This thermal energy heats the tissue to temperatures higher than the surrounding tissue resulting in a zone of ablation along the lesion path 140 due to thermal tissue necrosis. The temperatures of the tissue are preferably above the temperature where cellular/tissue necrosis occurs and the tissue is said to be ablated.

The shape of the lesion formed by the energy beam 110 depends on the characteristics of suitable combination factors such as the energy beam 110, the energy source 210 (including the material, the geometry, the portions of the energy source 210 that are energized and/or not energized, etc.), the energy source 210 characteristics, the electrical signal from the console 120 (including the frequency, the voltage, the duty cycle, the length and shape of the signal, etc.), and the characteristics of target tissue that the energy beam 110 propagates into and the speed at which the energy beam 110 is moved along the lesion path 140, as well as the characteristics of the target tissue including the thermal transfer properties and the ultrasound absorption, attenuation, and backscatter properties of the target tissue and surrounding tissue. The shape of the lesion path 140 and zone of ablation can be of any shape within the range of motion of the catheter 200. This includes but is not limited to linear, curvilinear, circular, spot, ellipsoid, freeform, etc.

The energy delivery system 100 of the preferred embodiments also includes a sensor 220 which may be either the energy source 210 or separate from the energy source 210 and may further function as a sensor to detect the distance of the tissue surface from the energy source 210, the thickness of the tissue, the characteristics of the tissue before, during, and after ablation, the incident beam angle, the relative motion of the tissue with respect to the energy source 210, and any other suitable parameter or characteristic of the tissue and/or the environment around the energy delivery system 100, such as the temperature. By detecting the information, the sensor 220 preferably functions to provide information which may be used to determine the lesion path 140, select and adjust the energy delivery and dose profile, identify collateral tissue, identify when a transmural lesion is created, and generally guide the planning, execution, and evaluation of the therapy.

The sensor 220 is preferably one of several variations. In a first variation, the sensor 220 is preferably an ultrasound transducer that has a substantially identical geometry as the energy source 210 to insure that the area diagnosed by the sensor 220 is substantially identical to the area to be treated by the energy source 210. More preferably, the sensor is the same ultrasound transducer as the ultrasound transducer of the energy source 210.

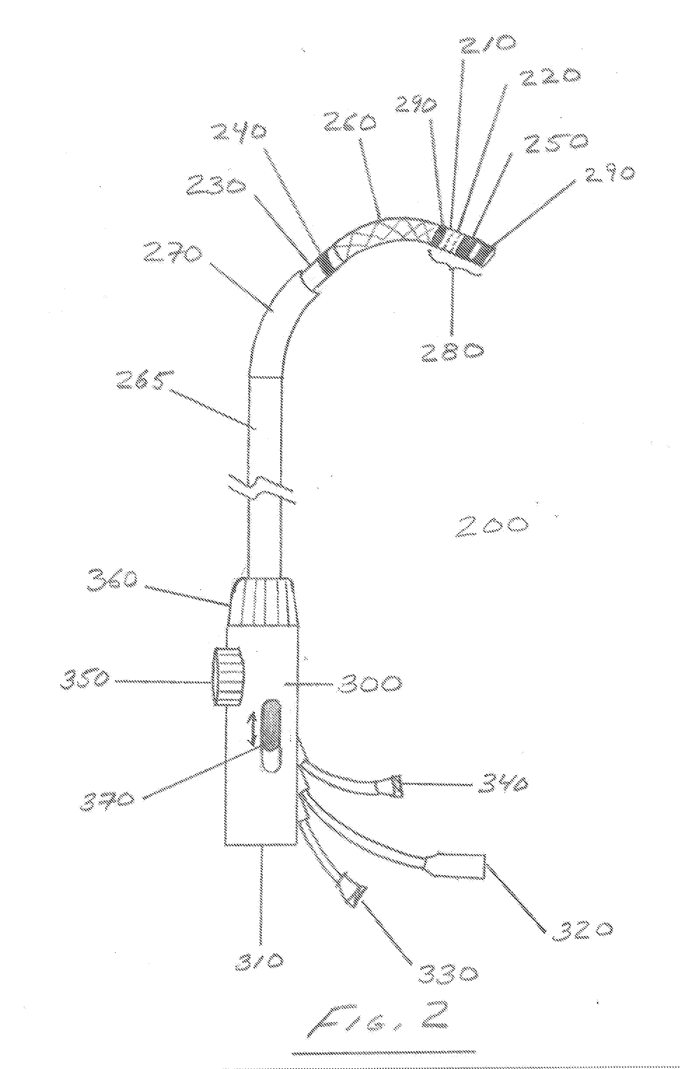

As shown in FIG. 2, the catheter 200 preferably consists of an inner shaft 230, an outer shaft 265, a handle 300, and the components thereof. The inner shaft 230 proximally (toward the physician) terminates near or within the handle 300. The distal region of the inner shaft 230 as shown, contains a proximal coil 240 for use with the mapping system. This proximal coil 240 may be located near the proximal area of the inner shaft deflecting section 260. Additional coils may be added as part of the inner or outer shafts (not shown). For example, an additional coil located proximal to the proximal coil 240 to further enable visualization of the outer shaft position or curvature of the outer shaft deflecting region 270. Distal to the inner shaft deflecting section 260 is the distal tip 280. The distal tip 280 houses the energy source 210 and the sensor 220. The energy source 210 and the sensor 220 are preferably ultrasound transducers and more preferably the same ultrasound transducer. A distal coil 250 for use with the mapping system is part of the distal tip 280. Irrigation of the energy source 210 and the sensor 220 may be accomplished by having a fluid lumen within the inner shaft 230. Irrigation fluid, such as saline, may be delivered through the fluid lumen via an inner shaft flush port 340 located in the handle. This fluid may be delivered under pressure, for example, using a pump or pressure cuff. In addition, the rate of fluid flow may be controlled by the console 120 or stand-alone pump or system.

The inner shaft 230 may have one or more radiopaque sections (e.g. the inner shaft deflecting section 260) or radiopaque markers identifying specific locations on the inner shaft 230 (e.g. a radiopaque marker band adjacent the proximal and/or the distal portion of the inner shaft deflection section 260).

Deflection of the inner shaft deflecting section 260 may be accomplished by connecting a portion of the inner shaft deflecting section 260, such as the distal region, to one or more control cables. These control cables may consist of pull wires, pull cables, pull fibers, or any other tension member, and be made of a variety of suitable materials, such as stainless steel, tungsten, carbon or aramid based fibers, etc. The control cables may be connected to the inner shaft deflecting section 260 by an attachment, such as a ring or bulkhead, and/or may be looped through a ring or bulkhead back to the proximal connection. The control cables may extend from distal region of the inner shaft deflecting section 260 back into the handle 300. Drive mechanisms within the handle 300, or catheter pod 150, may actuate these control cables. Actuation may be semi or fully automatic—moved by input from the controller to enable motion of the inner shaft deflecting section 260 and the distal tip 230, or manual (e.g. moved by the operator).

The inner shaft 230 may be moved within the outer shaft 265 by an actuator that is part of the handle 300 assembly, such as a slider 370. Movement of the slider 370 may be manual (e.g. operator), semi-automatic, or fully automatic—moved by input from the controller.

The inner shaft 230 and more preferably the distal tip 280 may include one or more electrical and/or radiopaque ring components 290, such as rings or “C” shaped elements that may also be in electrical connection to the handle 300 and to the console 120 or other system. These ring components 290 to be visualized, for example, under fluoroscopy or by other mapping systems and/or also provide electrical signals from the tissue if the rings are placed in contact with the tissue (e.g. to test for conduction block).

The outer shaft 265 consists primarily of a proximal region and a distally located outer shaft deflecting region 270. Deflection of the outer shaft deflecting region 270 may be may be accomplished by connecting a portion of the outer shaft deflecting region 270, such as the distal region, to one or more control cables. These cables may consist of pull wires, pull cables, pull fibers, or any other tension member, and be made of a variety of suitable materials, such as stainless steel, tungsten, carbon or aramid based fibers, etc. The control cables may be connected to the outer shaft deflecting region 270 by an attachment, such as a ring or bulkhead, and/or may be looped through a ring or bulkhead back to the proximal connection. The control cables may extend from distal region of the outer shaft deflecting region 270 back into the handle 300. Drive mechanisms within the handle 300, or catheter pod 150, may actuate these control cables, such as by rotation of the deflection knob 350. Actuation may be semi or fully automatic—moved by input from the controller to enable motion of the outer shaft deflecting region 270, or manual (e.g. moved by the operator using deflection knob 350).

The outer shaft 265 may be moved with respect to the inner shaft 230. For example, rotation of the outer shaft may be accomplished by having the outer shaft 265 in connection to a rotation knob 360, wherein rotation of the rotation knob 360 causes the outer shaft 265 to rotate with respect to the inner shaft 230. This rotation may be accomplished irrespective of the position of the inner shaft 230 in relation to the outer shaft 265 or the handle 300, e.g. the inner shaft 230 may be fully advanced, fully retracted, or any position in between with respect to the handle 300 and outer shaft 265. If desired, a fluid column or irrigation of the area between the inner shaft 230 and the outer shaft 265 may be accomplished by having a fluid, such as saline, delivered through the area via an outer shaft flush port 330 located in the handle. This fluid may be delivered under pressure, for example, using a pump or pressure cuff. In addition, the rate of fluid flow may be controlled by the console 120 or stand-alone pump or system.

The outer shaft 265 may have one or more radiopaque sections (e.g. the outer shaft deflecting region 270) or radiopaque markers identifying specific locations on the outer shaft 265 (e.g. a radiopaque marker band adjacent the distal end of the outer shaft deflecting region 270). The outer shaft may include one or more coils for position location information.

The handle 300 is connected to the inner shaft 230 and the outer shaft 265, as well as incorporating a catheter pod connection 310 back to the console 120. The catheter pod connection 310 may go to a catheter pod 150, which may incorporate one or more motors to control actuation of inner and/or outer shaft components (e.g. control cables) as well as connection to some or all of the electrical components (e.g. the energy source 210, sensor 220, etc). Connection between the catheter pod connection 310 and the catheter pod 150 may incorporate a sterile adapter 160 to form a sterile barrier. The sterile adapter 160 may also include an integral or separate cover that extends over at least a portion of the catheter pod 150.

The handle 300 may provide connections from the coil or coils of the inner shaft 230 and/or outer shaft 265 via the connector 320 to the console 120 or a stand-alone system. This connector 320 may also incorporate an electrical pathway for the ring components 290 to connect to the console 120 or other stand-alone system.

The energy delivery system 100 of the preferred embodiments also includes a console 120 (illustrated in FIG. 1) coupled to the sensor 220. The console 120 preferably is operatively coupled to the catheter 200, which may be through the catheter pod 150 and the sterile adapter 160. In addition, the mapping system may be integrated with the console 120 or may be stand alone. The mapping system may include a window field generator 170 and a reference sensor 180. The detection device 400 may also be coupled to the console 120 or mapping system.

The console 120 may provide an integrated or stand-alone display for providing information to the operator as well as providing a user interface for the operator in input information and/or control at least a portion of the ablation system 100 and the procedure. Displayed information can included but is not limited to the procedure workflow which may include 2D and/or 3D maps of the distance of the tissue surface from the energy source 210, the thickness of the tissue, a lesion path 140, energy, dose profile, and temperature parameters, angularity of the distal tip 130 and energy beam 110 in relation to the tissue, tissue motion characteristics, the characteristics of the ablated tissue, information related to collateral tissue, position of the mapping system components, including the field generator 170, reference sensor 180, detection device 400, system operating conditions, patient information, and any other suitable parameter or characteristic of the tissue and/or the environment around and including the energy delivery system 100. In addition, these may also be acted upon and provide the capability for the operator to control, move, modify or adjust what and how information is displayed as well as any or all input parameters related to the procedure. The operator inputs and user interface may be for example a touch screen display, a display module, a keyboard, etc.

The console is preferably connected to electrical power via a plug 190 which may use the typical wall power available at the site of operation.

The console 120 may comprise a console-integrated or stand-alone mapping system that enables precise control of the position of the distal tip 130 of the catheter 200, enables position and pointing information of the distal tip 130 and energy beam 110, and position information from the detection device. All of this information may be shown on a display and used as part of the procedure.

The console 120 preferably controls manipulation of the inner shaft deflecting section 260 to enable accuracy of motion of catheter 200 components and the energy beam 110. The console 120 controls manipulation of the inner shaft deflecting section 260 through drive mechanisms and optionally incorporates sensors in or near the handle 300. The console 120 directs movement of these drive mechanisms according to mathematical (algorithmic) models that predict the distal deflection and/or motion in response to movement of the drive mechanisms. These models of the mechanical transfer function may be imperfect, and may result in motion of the inner shaft deflecting section 260 that deviates from the intended motion, even with feedback provided from sensors reading the drive mechanisms or control cables. Distal tip 230 position distortion may be reduced by using the coils and console 120 and/or integrated or stand-alone mapping system to provide positional information of the distal tip 230 and adjust and modify the actions of the drive mechanisms to correct for any distortion introduced along the catheter 200. In effect, the position data from the mapping system may be used to provide dynamic feedback in a closed or semi-closed loop control manner.

This accuracy of motion of the inner shaft deflecting section 260 is important as it is used to direct the energy beam 110 during multiple aspects of a typical procedure. This motion includes but is not limited to scanning or mapping and trajectories as part of pre-ablation, ablation, and evaluation. The inner shaft deflecting section 260 may be commanded to perform a scan (e.g. a spiral) of an area of interest (e.g. the left atrium 10). This is accomplished by inputs on the console 120 initiating ultrasound and having the drive mechanisms in the handle 300 deflect the inner shaft deflecting section 260 to steer the distal tip 230 and energy beam 110 in a specific pattern to obtain ultrasound information of the tissue and positional information, without the energy source 210 being in contact with the tissue. This information may include distance to tissues, thickness of the tissues, collateral tissues (e.g. esophagus), tissue characteristics, tissue motion, angle of the energy beam 110 to the tissues, etc. Positional information may be individually or a combination of drive mechanism and/or control cable position/movement, coil position, etc. The positional and tissue information may be shown on the display, in 2D or 3D, and may include distance from the distal tip 230 or energy source 210 to the tissue surface, thickness of the tissues, collateral tissues, tissue characteristics, tissue motion, angle of the energy beam 110 to the tissues, etc.

After a lesion path 140 has been selected by the operator, which may include appropriately placing the lesion path 140 such that it does not cross any tissue or anatomical structure that is sensitive to ablation, sensitive to overheating, or any other characteristic that may require special treatment during the ablation process, including, but not limited to esophageal tissue and nerves such as the phrenic nerve, the inner shaft deflecting section 260 may be commanded to perform a pre-ablation trajectory or scan. This pre-ablation trajectory follows the specified lesion path 140, to gather additional information as described above.

Once sufficient data is gathered, the energy delivery system 100 may recommend a dose profile along the lesion path 140. It is likely this dose profile is variable along the lesion path 140 due to tissue thickness, beam angle of incidence, tissue motion, collateral tissue, etc. The operator then may move or adjust the position of the lesion path 140 and/or adjust the recommended dose profile. It may be preferable when there is no collateral tissue within a portion of the lesion path, that a higher dose profile be selected in that region to ensure transmurality of the lesion. Where there is collateral tissue that is undesirable to heat or ablate, a lower dose profile may be selected in that region for safety reasons. Additional scans may be conducted at any time. When the operator is satisfied with the lesion path and dose profile, the inner shaft deflecting section 260 is commanded to perform the ablation trajectory, which delivers the dose profile along the lesion path to ablate the target tissue.

During the ablation, real-time information is gathered including but not limited to position, operating parameters, and characteristics of the tissue that may include degree of necrosis, transmurality of the lesion, etc. This information may be displayed to the operator.

In addition, the inner shaft deflecting section 260 may be commanded to perform an evaluation trajectory or scan. This may comprise following the lesion path 140 or performing a scan of a larger region of tissue to obtain and/or characterize tissue information, including degree of necrosis, transmurality of the lesion, etc. This information may be displayed to the operator.

The console 120 preferably controls the energy beam 110 emitted from the energy source 210 by modifying the electrical signal sent to the energy source 210, such as the frequency, the voltage, the duty cycle, the length of the pulse, and/or any other suitable parameter. Additionally, the console 120 may further be coupled to or have an integrated fluid flow controller for controlling the rate of fluid flow through one or more of the catheter 200 fluid lumens. The console 120 may control the fluid flow controller to increase or decrease fluid flow based on if the energy delivery system 100 is in mapping mode or in therapy mode, the characteristics of the target tissue or ablated tissue, the temperature of the tissue and/or energy source 210, and/or the characteristics of any other suitable or energy delivery system 100 measured or monitored condition.

By controlling the energy beam 110 (and/or the cooling of the targeted tissue or energy source 210), the zone of ablation is controlled. For example, the depth of the zone of ablation is preferably controlled such that a transmural lesion is achieved. Additionally, the console 120 preferably functions to minimize the possibility of creating a lesion beyond the targeted tissue, for example, beyond the outer atrial wall. If the energy source 210 and/or sensor 220 detects the lesion extending beyond the outer wall of the atrium or that the depth of the lesion has reached or exceeded a preset depth, the console 120 preferably appropriately decreases or terminates the energy delivery and dose. Conversely, if the zone of ablation is of insufficient depth, the console 120 may increase the energy delivery and dose to attain a transmural lesion. Functions of the console 120 may be in response to inputs from the operator, semi-automated, or fully automated including specifying all the operating parameters and outputs to the catheter pod 150 and catheter 200.

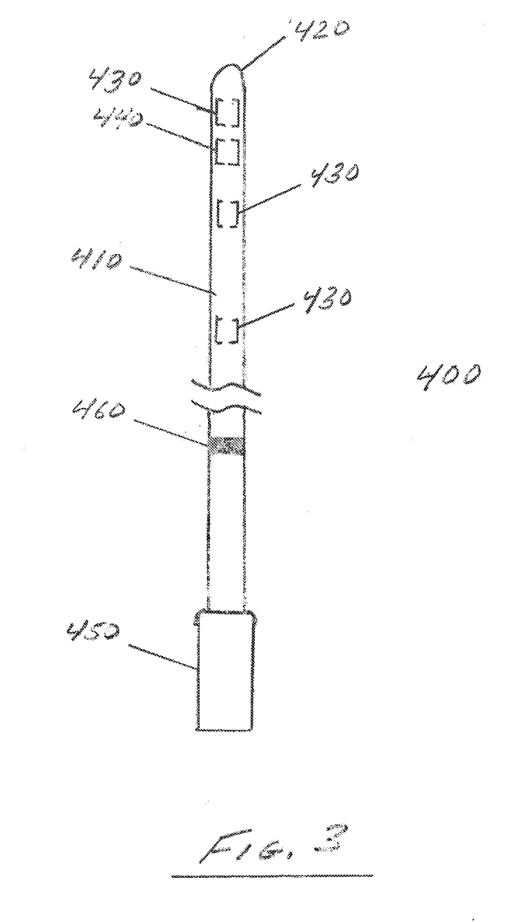

The energy delivery system 100 may include a detection device 400. As shown in FIG. 3, the detection device is preferably an esophageal probe that is inserted into the esophagus 20 and interacts with the console 120, console-integrated, or stand-alone mapping system. The detection device 400 may provide multiple functions (e.g. position information, temperature information, etc.). As shown in FIG. 3, the detection device 400 comprises a detection device shaft 410 which may extend from the detection device connector 450 to the detection device tip 420. The detection device shaft 410 may be constructed of any suitable material including but not limited to thermoplastic and thermoset polymers, including polyvinyl chloride, urethane, nylon, polyethylene, co-polymer blends, etc. The detection device 400 may optionally contain one or more thermistors 440 or thermocouples for monitoring temperature of the tissue or area within the vicinity of the thermistors 440 or thermocouples, as well as optionally, one or more detection device coils 430 which work in conjunction with the console 120 and/or mapping system. A preferred embodiment optionally may comprise four detection device coils 430 and optionally no thermistors or thermocouples. In the proximal region, there is a detection device connector 450 that enables connection of the detection device 400 to the console 120 and/or mapping system. The detection device 400 terminates distally in a detection device tip 420 that is preferably atraumatic. The detection device shaft 410 may contain one or more marker bands 460 that provide an indication of the distance from the marker band 460 to the detection device tip 420. There may also be one or more marker bands 460 located near the detection device coil(s) 430, the thermistor(s) 440 or thermocouple(s), and/or the detection device tip 420. Any or all of the marker bands 460 may be radiopaque. Additionally the detection device may contain a lumen at least partially through the detection device. The detection device may also contain a balloon. The lumen and/or balloon may be filled with a fluid such as saline, water, gas (e.g. carbon dioxide, air). Liquids such as saline or water are preferably filled with microbubbles to enhance echogenicity. The energy delivery system may enable sensing the filled lumen or balloon with an ultrasound signal delivered by the energy source.

The detection device 400 may move within a specific range (e.g. within the esophagus 20) to obtain positional information that is provided to the operator. This movement allows the console 120/mapping system to display the position of the detection device 400 or elements of the detection device 400 (e.g. detection device coil 430) and construct or infer and show the position of the esophagus 20, or portions of, or a position or positions within the esophagus in conjunction with other tissues (e.g. all or a portion of the left atrium 10, the target tissue, etc.) on a display, in 2D or 3D, to assist the operator in properly positioning and/or adjusting the lesion path 140 and/or dose profile.

The detection device may be positioned and left in place with one or more detection device coils 430 and or one or more thermistors 440 or thermocouples enabling discrete or real-time monitoring of the position and or temperature.

The detection device 400 may be positioned and/or repositioned at any time such that the thermistor(s) 440 or thermocouple(s) are appropriately placed to monitor temperature of a tissue (e.g. the esophagus 20), during any part of the procedure, including before, during, and/or after ablation.

The detection device 400 may provide positional information to the energy delivery system 100 that is used in conjunction with the ultrasound signal to provide additional information as to the position or structures of a collateral tissue. For example, detection device coils 430 may provide a location of the detection device coils within a collateral tissue, and then that location is used to identify one or more structures in the ultrasound signal to locate the wall and or components of the collateral tissue which then may be shown on the display.

One example of use of the energy delivery system 100 for ablating tissue within the left atrium 10 is described. Steps may be repeated, occur in different order, or eliminated depending on the desired procedure workflow. Exemplary steps include, but are not limited to:
1) Transseptal access is gained through standard percutaneous techniques.
2) A sheath 500 is inserted into the patient with the distal end crossing the septum to provide access to the left atrium 10.
3) The distal portion of the catheter 200 is placed within the sheath 500 and advanced until the distal tip 280 exits the distal end of the sheath 500.
4) The catheter 200 is advanced further until the outer shaft deflecting region 270 is adjacent the distal end of the sheath 500.
5) The outer shaft deflecting region 270 may be deflected using the deflection knob 350, rotated using the rotation knob 360, and/or the catheter 200 may be advanced or retracted to grossly align the distal tip 230 pointing at the region of interest (e.g. the left hemisphere of the left atrium 10).
6) The use of the catheter 200 coils and console 120/mapping system can be used along with fluoroscopy to enable this positioning.
7) The inner shaft 230 is advanced using the slider 370 until the inner shaft deflecting section 260 extends out of the outer shaft 265.
8) The detection device 400 is placed within the esophagus 20 and advanced until a detection device coil 430 is grossly aligned with the left atrium. This may be accomplished using fluoroscopy and/or by use of the detection device coil 430 or marker band 460.
9) The detection device 400 is moved (advanced/retracted) within the region of the left atrium while position information is collected by the console 120/mapping system. Additionally, the detection device may have multiple detection device coils 430 which may be positioned spanning the region of interest, in this case the left atrium, and left in position, or moved, while discrete or real-time positional information is collected by the console 120/mapping system.
10) The operator, using the console 120, directs the energy ablation system 100 to perform a scan (e.g. a spiral) of the left hemisphere of the left atrium 10. This is accomplished by inputs on the console 120 initiating ultrasound and having the drive mechanisms in the handle 300 deflect the inner shaft deflecting section 260 to steer the distal tip 230 and energy beam 110 in a specific pattern to obtain ultrasound information of the tissue and positional information, without the energy source 210 being in contact with the tissue. This information may include distance to tissues, thickness of the tissues, collateral tissues (e.g. esophagus), tissue characteristics, tissue motion, angle of the energy beam 110 to the tissues, etc. Positional information may be obtained individually or through a combination of drive mechanism and/or control cable position/movement, coil position, etc.
11) The positional and tissue information is shown on the display, such as distance from the distal tip 230 or energy source 210 to the tissue surface, thickness of the tissues, collateral tissues, tissue characteristics, tissue motion, angle of the energy beam 110 to the tissues, etc. This may be displayed in 2D or preferably 3D.
12) The operator then draws a desired lesion path 140 on the display. This may be done using a touch screen, keyboard, mouse, control stick, etc.
13) The operator commands the energy delivery system 100 to follow the drawn lesion path 140 in a pre-ablation scan. During this movement along the lesion path 140, detailed information is gathered about the tissue as described in step 10. The energy delivery system may then provide the operator with a suggested dose profile along the lesion path 140 based on all the available information.
14) If the lesion path crosses any tissue or anatomical structure that is sensitive to ablation, sensitive to overheating, or any other characteristic that may require special treatment during the ablation process, including, but not limited to esophageal tissue and nerves such as the phrenic nerve, the lesion path may be moved to exclude this tissue (e.g. click with a mouse on a portion of the lesion path 140 shown on the display and drag any portion or all of the lesion path to a new position). Step 13 may be repeated prior to ablation.
15) If it is not possible to move the lesion path to exclude the tissue or anatomical structure not desired to be ablated, then a faster speed during ablation, lower beam energy, extra tissue sensing, or any other suitable alterations to the ablation process may be commanded by the operator and/or console 120.
16) Where there is no tissue or anatomical structure that is not desired to be ablated along the lesion path 140, a slower speed during ablation and/or higher beam energy may be commanded by the operator and/or console 120 to ensure transmurality of the lesion.
17) Upon confirming the lesion path and ablation parameters are appropriate to initiate ablation, the operator commands the energy delivery system 100 to create the lesion along the lesion path 140. During the ablation trajectory, real-time information is gathered about the characteristics of the tissue that may include degree of necrosis, transmurality of the lesion, etc. This information may be displayed to the operator.
18) After the ablation is complete, the operator may command the energy delivery system 100 to perform an evaluation trajectory or scan. This may comprise following the lesion path 140 or performing a scan of a larger region of tissue to determine additional tissue information.
19) Additional scans and ablations may be conducted in that region. A second ablation on a different lesion path 140 around the same pulmonary vein or veins may be conducted to improve efficacy (e.g. an ellipse inside an ellipse around both left pulmonary veins).
20) The operator may then proceed to grossly align the distal tip 230 pointing at another region of interest (e.g. the right hemisphere of the left atrium 10) and repeat the procedure as described above to create an additional lesion.
21) Following isolation around all the pulmonary veins, additional lesions may be created such as a roof line, an isthmus line, or the sheath 500 and catheter 200 may be repositioned to create lesions in the right atrium.

Detecting and compensating for collateral tissue and the procedural workflow described above may include the step of identifying the phrenic nerve. Identifying the phrenic nerve may include positioning the catheter 200 to enable a scan of the region of interest which includes the phrenic nerve. This scan would be used to generate and display the position of or the likely position of the phrenic nerve on the display. Additionally, mechanical and/or electrical stimulation of the phrenic nerve and monitoring the patient for a reflex or other identifiable response to the stimulation may be used to identify and display the position of or the likely position of the phrenic nerve on the display.

Mechanical force stimulation of the phrenic nerve is preferably conducted by having the energy delivery device 100 deliver the mechanical force as an ultrasound pulse or series of pulses to elicit the desired response. The ultrasound pulse is preferably a short-duration high-intensity signal, with the resulting pressure wave mechanically stimulating the phrenic nerve. The ultrasound energy delivered may be a series of pulses, a high or low frequency signal, or any other suitable ultrasound signal, sufficient to stimulate the phrenic nerve and provide information as to its position.

Electrical stimulation of the phrenic nerve will similarly elicit a response which may include a nerve signal, muscle contraction, or any other suitable internal or external response that may be monitored.

The energy delivery system 100 and detection device 400 may be used to create a zone of ablation in the ventricle. Similar steps may be employed to identify collateral tissue and minimize ablating or otherwise damaging the collateral tissue and to increase the likelihood of procedure success. It may be desirable to create a zone of ablation that is not transmural. For example, in treating ventricular tachycardia or periventricular contractions, it may be sufficient to only ablate an electrical channel and or a region of scar tissue to achieve the desired therapeutic effect.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for ablating tissue, said method comprising:
providing a catheter having an ultrasound transducer adjacent a distal end thereof and a sensor adjacent the ultrasound transducer;
positioning the ultrasound transducer and the sensor adjacent target tissue;
sensing collateral tissue adjacent the target tissue;
determining a lesion path based on the target tissue and the collateral tissue; and
ablating the target tissue along the lesion path with a beam of ultrasound from the ultrasound transducer thereby forming a continuous lesion in the target tissue.

2. The method of claim 1, wherein the ultrasound transducer and the sensor are the same ultrasound transducer.

3. The method of claim 1, further comprising maintaining a gap between the ultrasound transducer and the target tissue so that the ultrasound transducer is not in contact with the target tissue during energy delivery.

4. The method of claim 1, wherein the sensing of the collateral tissue comprises identifying collateral tissue with the ultrasound transducer or the sensor.

5. The method of claim 1, wherein the sensing of the collateral tissue comprises placing an esophageal probe in the esophagus.

6. The method of claim 5, wherein the esophageal probe comprises a plurality of electromagnetic coils.

7. The method of claim 5, wherein the esophageal probe is identified by the ultrasound transducer or sensor.

8. The method of claim 5, further comprising moving the esophageal probe within the esophagus to obtain position information.

9. The method of claim 5, wherein the esophageal probe contains an electromagnetic coil.

10. The method of claim 5, wherein the esophageal probe electromagnetic sensor can be identified by a system.

11. A method for ablating tissue, said method comprising:
providing an ablation system, the ablation system having mapping and ablation capability, and
providing a catheter having an ultrasound transducer adjacent a distal end thereof and a sensor adjacent the ultrasound transducer;
positioning the ultrasound transducer and the sensor adjacent target tissue;
mapping the target tissue;
determining a position of collateral tissue;
determining a lesion path based on the target tissue and the collateral tissue; and
ablating the target tissue along the lesion path with a beam of ultrasound from the ultrasound transducer thereby forming a continuous lesion in the target tissue.

12. The method of claim 11, wherein the ultrasound transducer and the sensor are the same ultrasound transducer.

13. The method of claim 11, further comprising maintaining a gap between the ultrasound transducer and the target tissue so that the ultrasound transducer is not in contact with the target tissue during energy delivery.

14. The method of claim 11, wherein the ablation system has a graphical display, the method further comprising displaying information on the graphical display.

15. The method of claim 14, wherein the graphical display shows the target tissue.

16. The method of claim 14, wherein the graphical display shows the collateral tissue.

17. The method of claim 14, wherein the graphical display shows the thickness of the target tissue.

18. The method of claim 11, further comprising determining a dose profile delivered to ablate the target tissue based on the target tissue thickness.

19. The method of claim 11, further comprising determining a dose profile delivered to ablate the target tissue based on the position of the collateral tissue.

20. The method of claim 11, further comprising determining a dose profile delivered to ablate the target tissue based on a depth of the target tissue.

21. The method of claim 11, wherein determining the position of the collateral tissue comprises identifying the collateral tissue with the ultrasound sensor or the sensor.

22. The method of claim 11, wherein the sensing of the collateral tissue comprises placing an esophageal probe in the esophagus.

23. The method of claim 22, further comprising identifying the esophageal probe with the ultrasound transducer or sensor.

24. The method of claim 22, wherein the esophageal probe contains an electromagnetic coil.

25. The method of claim 24, wherein the esophageal probe contains a plurality of electromagnetic coils.

26. The method of claim 22, wherein the esophageal probe comprises an electromagnetic coil, the method further comprising identifying the esophageal probe with the ablation system.

27. The method of claim 11, wherein the ablation system contains an electromagnetic positioning system.

28. A system for ablating tissue, said system comprising:
an ablation system comprising a sensor adapted to detect collateral tissue adjacent the target tissue, and
wherein the sensor provides feedback to the ablation system to provide positional information related to the collateral tissue; and
a catheter having an ultrasound transducer adjacent a distal end thereof,
wherein the ultrasound transducer is adapted to deliver a beam of ultrasound energy to target tissue thereby ablating the target tissue and forming a continuous lesion in the target tissue.

29. The system of claim 28, wherein the ultrasound transducer and the sensor are the same ultrasound transducer.

30. The system of claim 28, wherein the beam of ultrasound energy is a collimated beam of ultrasound energy.

31. The system of claim 28, wherein the ultrasound transducer is disposed away from the target tissue such that the ultrasound transducer is not in contact with the target tissue during energy delivery.

32. The system of claim 28, wherein the ablation system comprises a graphical display.

33. The system of claim 32, wherein the graphical display shows the target tissue.

34. The system of claim 32, wherein the graphical display shows the collateral tissue.

35. The system of claim 32, wherein the graphical display shows the thickness of the target tissue.

36. The system of claim 28, wherein a dose profile delivered to ablate the target tissue is determined by the tissue thickness.

37. The system of claim 28, wherein a dose profile delivered to ablate the tissue is determined by the position of the collateral tissue.

38. The system of claim 28, wherein a dose profile delivered to ablate the tissue is determined by a depth of the target tissue.

39. The system of claim 28, wherein the ultrasound transducer or the sensor is configured to identify the collateral tissue.

40. The system of claim 28, further comprising an esophageal probe configured to be disposed in the esophagus, the esophageal probe configured to facilitate sensing of the collateral tissue.

41. The system of claim 40, wherein the esophageal probe contains a plurality of electromagnetic coils.

42. The system of claim 40, wherein the esophageal probe can be identified by the ultrasound transducer.

43. The system of claim 40, wherein the esophageal probe comprises an electromagnetic coil.

44. The system of claim 40, wherein the esophageal probe electromagnetic coil can be identified by the ablation system.

45. The system of claim 28, wherein the ablation system comprises an electromagnetic system.

46. The system of claim 45, wherein the electromagnetic coil can be identified by the electromagnetic system.
